(19) United States
(12) Patent Application Publication
(10) Pub. No.: US 2022/0195389 A1
(43) Pub. Date: Jun. 23, 2022

(54) METHODS OF IMPROVING THE LONGEVITY OF IMMUNE CELLS

(71) Applicant: University of Houston System, Houston, TX (US)

(72) Inventors: Navin Varadarajan, Houston, TX (US); Irfan Naseem Bandey, Houston, TX (US); Melissa Martinez Paniagua, Crosby, TX (US)

(73) Assignee: University of Houston System, Houston, TX (US)

(21) Appl. No.: 17/604,714

(22) PCT Filed: Apr. 17, 2020
(86) PCT No.: PCT/US20/28727

§ 371 Date: Oct. 18, 2021

Related U.S. Application Data
(60) Provisional application No. 62/835,131, filed on Apr. 17, 2019.

(51) Int. Cl. C12N 5/0783; C07K 14/725; C07K 16/28; C12N 9/12

(52) U.S. Cl. CPC C12N 5/0638; C07K 14/7051; C12N 2501/727; C12N 9/1205; C12N 2510/00; C07K 16/2803

(57) ABSTRACT

Embodiments of the present disclosure pertain to methods of modifying immune cells by increasing the AMP-activated protein kinase (AMPK) activity of the immune cells in order to produce modified immune cells with enhanced longevity. In some embodiments, the methods of the present disclosure include one or more of the following steps: (1) obtaining immune cells from a subject; (2) increasing the AMPK activity of the immune cells; (3) expanding the modified immune cells; (4) introducing the modified immune cells to a subject; and (5) treating a disease in the subject. In further embodiments, the methods of the present disclosure include an in vivo method of increasing the AMPK activity of immune cells in a subject in order to treat a disease in the subject. Additional embodiments of the present disclosure pertain to the modified immune cells.

### CROSS-REFERENCE TO RELATED APPLICATIONS

This application claims priority to U.S. Provisional Patent Application No. 62/835,131, filed on Apr. 17, 2019. The entirety of the aforementioned application is incorporated herein by reference.

### BACKGROUND

Methods of improving the longevity of immune cells in vivo to fight disease is a major unsolved problem. Embodiments of the present disclosure address this problem.

### SUMMARY

In some embodiments, the present disclosure pertains to methods of modifying immune cells. In some embodiments, the methods of the present disclosure include a step of increasing the AMP-activated protein kinase (AMPK) activity of the immune cells to produce modified immune cells with enhanced longevity. In some embodiments, the methods of the present disclosure include one or more of the following steps: (1) obtaining immune cells from a subject; (2) increasing the AMPK activity of the immune cells; (3) expanding the modified immune cells; (4) introducing the modified immune cells to a subject; and (5) treating a disease in the subject. In further embodiments, the methods of the present disclosure include an in vivo method of increasing the AMPK activity of immune cells in a subject in order to treat a disease in the subject. Additional embodiments of the present disclosure pertain to modified immune cells with increased AMPK activity that enhances the longevity of the modified immune cells.

The AMPK activity of immune cells may be increased in various manners. For instance, the increasing occurs by introducing a gene that encodes a protein into the immune cells. In some embodiments, the introduced protein includes, without limitation, AMPK, Sirtuins, serine/threonine-protein kinase (STK11), CD36, trehalose transporter, derivatives thereof, fusion proteins thereof, subunits thereof, and combinations thereof. In some embodiments, the introduction of proteins into immune cells includes introducing a gene that encodes a protein into the immune cells. In some embodiments, the introduction of proteins into immune cells includes introducing an expression vector into the immune cells. In some embodiments, the expression vector encodes and expresses the protein in the immune cells.

In some embodiments, the AMPK activity of immune cells is increased by exposing the immune cells to an activator for at least 12 hour or at least 24 hours. In some embodiments, the activator includes, without limitation, an AMPK activator, an STK11 activator, a Sirtuin activator, a CD36 activator, a Klotho activator, an autophagy activator, doxycycline, and combinations thereof.

The enhanced longevity of the modified immune cells of the present disclosure can be determined by various factors. For instance, in some embodiments, such factors include, without limitation, enhanced motility, persistent motility, enhanced cellular polarity, enhanced respiratory capacity, an enhanced number of punctate mitochondria, increased mitochondrial mass, reduced conjugation durations, enhanced spare respiratory capacity (SRC), enhanced capacity for oxidative metabolism, and combinations thereof.

The methods of the present disclosure can be utilized to enhance the longevity of various immune cells by increasing the AMPK activity of the immune cells. For instance, in some embodiments, the immune cells include lymphocytes. In some embodiments, the lymphocytes include, without limitation, T-cells, NK-cells, B-cells, and combinations thereof. In some embodiments, the immune cells include lymphocytes that have been engineered to express one or more immune receptors, such as chimeric antigen receptors (CAR) that target CD19.

The methods and modified immune cells of the present disclosure can be utilized to treat various diseases. For instance, in some embodiments, the disease to be treated by the modified immune cells of the present disclosure is a type of cancer. In some embodiments, the subject is a human being suffering from the cancer. In some embodiments, the subject is undergoing cancer immunotherapy.

In some embodiments, the treatment first includes modifying immune cells in vitro in accordance with the methods of the present disclosure and then administering the modified immune cells to the subject. In some embodiments, the treatment also includes expanding the modified immune cells in vitro prior to administration. In some embodiments, the treatment includes modifying immune cells in vivo in accordance with the methods of the present disclosure.

### DESCRIPTION OF THE DRAWINGS

FIG. 1A illustrates a method of modifying immune cells.

FIG. 1B illustrates an in vitro method of modifying immune cells for disease treatment in a subject.

FIG. 1C illustrates an in vivo method of modifying immune cells for disease treatment in a subject.

FIG. 2 illustrates integrated profiling of T-cell polyfunctionality at the single-cell level. FIG. 2A provides a representative example of a polyfunctional 19-28z T cell that participated in serial killing and secreted IFN-γ. Timelapse imaging microscopy in nanowell grids (TIMING) is utilized to quantify T-cell intrinsic behavior like persistent motility and the kinetics of the interaction leading to induction of apoptosis within tumor cells. At the conclusion of the TIMING assay, the IFN-γ molecules captured onto the beads during TIMING are revealed by using appropriate fluorescently labeled antibodies. Scale bar is 20 km. FIG. 2B provides a schematic description of parameters measured in TIMING experiments. FIG. 2C provides cumulative contact duration between effector and tumor cells leading to different functional outcomes. Effector cells that only secrete IFN-γ (monofunctional) exhibited longer contact duration compared to cells that kill one or serial irrespective of whether that secrete IFN-γ. The black arrow indicates a subpopulation of T cells that were conjugated to the tumor cells for the entire period of observation. FIG. 2D provides kinetics of killing based on t_{Contact} and t_{Death} of serial killer and single killer T cells for subsets of effector that participate in killing. FIG. 2E provides motility of the different functional subsets of 19-28z T cells. All data in FIGS. 2C-E are derived from analyzing 1,178 nanowells containing one T-cell and 2-5 tumor cells, and one or more beads. All p values for all multiple comparisons were computed using Kruskal-Wallis non-parametric tests and each dot represents a single effector cell.

FIG. 3 provides data relating to the enrichment and functional characterization of motile 19-28z T cells. FIGS. 3A and 3B show comparisons between the persistent motility (FIG. 3A) and aspect ratio (FIG. 3B) of polarization of migrated (motile) and non-migrated (non-motile) cells. Error bars represent 95% CI. FIG. 3C shows sustained killing mediated by individual motile 19-28z T cells ordered by the encounter with tumor cells. Error bars indicate 95% CI. FIG. 3D shows a state transition diagram illustrating the evolution of the interaction between 19-28z T cells and tumor cells within single-cell assays. The thickness of the lines connecting the state is proportional to the frequency of the transition. The data was obtained from nanowells containing exactly one T cell and 3-5 tumor cells. All p values were computed using Mann-Whitney tests and each dot represents a single effector cell. All data shown here are from one representative population derived from at least three independent healthy donor-derived 19-28z T cells.

FIG. 4 shows that the profile of motile 19-28z T cells is consistent with naïve-like T cells. FIGS. 4A and 4B show that the phenotype (FIG. 4A) and GzB expression (FIG. 4B) of the motile and unsorted 19-28z T cells as determined by flow cytometry. FIG. 4C shows a volcano plot depicting the differentially expressed genes (DEGs) that discriminate between motile and unsorted 19-28z T cells, as determined by RNA-seq on six paired populations of motile and unsorted 19-28z T cells. FIG. 4D shows that the GSEA-derived enriched C2 and C7 curated pathways were plotted using the enrichment map application in Cytoscape using a cutoff FDR q-value=0.25 and a p-value=0.05. Nodes (circles colored red) represent pathways and the edges (green lines) represent the overlapping gene among pathways. The size of nodes represents the number of genes enriched within the pathway and the thickness of edges represents the number of overlapping genes. FIG. 4E shows GSEA enrichment plot derived from pre-ranked DEGs comparing motile 19-28z and unsorted populations. FIG. 4F shows basal OCR levels measured for the different 19-28z T cell populations. P-value is for comparison of the SRC comparing the motile and non-motile subsets using multiple t tests. FIGS. 4G-H show the motility and polarization of 19-28z T cells treated with Compound C (CC). All data representative of at least three independent experiments performed with cells from at least three healthy human donors derived 19-28z T cells, and plotted as mean±SEM.

FIG. 5 shows that motile 19-28 T cells reject established leukemia and sustain persistence in vivo. FIG. 5A shows false-colored images illustrating the photon flux from ffLuc expressing EGFP⁺NALM-6 cells. FIG. 5B shows time course of the longitudinal measurements of NALM-6 derived photon flux from the three separate cohorts of mice (n=10 in each group). The background luminescence was defined based on mice with no tumor. Error bars represent SEM and p values are computed using the Mann-Whitney test. FIG. 5C shows that, on day 31, four mice from each group were euthanized, and tissues (bone marrow and spleen) were harvested and analyzed by flow cytometry for expression of human CD3 (human T cells) and EGFP (gated within hCD19 cells). The CAR⁺ T cells were identified by a scFv-specific antibody, as described previously. FIG. 5D shows false-colored images illustrating the photon flux from ffLuc expressing EGFP⁺NALM-6 cells treated with suboptimal doses of 19-28z T cells. FIG. 5E shows time course of the longitudinal measurements of NALM-6 derived photon flux from the three separate cohorts of mice (n=5 in each group). The background luminescence was defined based on mice with no tumor. Error bars represent SEM and p values are computed using a two-way ANOVA.

FIG. 6 shows the quantification of the link between motility and functionality in diverse CARs. FIG. 6A shows a schematic illustrating the CAR structure, manufacturing and expansion, and the target cells used for profiling functionality of individual CAR⁺ T cells using TIMING. FIG. 6B illustrates the persistent motility of individual killer and non-killer CAR T cells without and with conjugation to tumor cells. All data from an E:T of 1:1, and error bars represent 95% CI. All p values were computed using Mann-Whitney tests and each dot represents a single effector cell.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The activity of immune cells is essential for fighting disease. Along with antibody immunotherapy, genetically engineering T cells for redirecting immune responses is a promising frontier in cancer treatment, especially with several clinical trials approaching commercialization of cell-based drugs.

Adoptive cell therapy (ACT), based on infusing in vitro expanded T-cells bearing either T-cell receptors (TCR), or chimeric antigen receptors (CAR), have demonstrated dramatic and durable responses, even in heavily pretreated patients. Despite the promise, since current pharmaceutical manufacturing paradigms are optimized for small molecules, and more recently proteins, the infusion of T-cells as drugs presents unique challenges, as recognized by the U.S. Food and Drug Administration (FDA). One of the major challenges associated with immunotherapies has been an incomplete understanding of the comprehensive role of T-cell metabolism in enabling anti-tumor efficacy.

The persistence of cells in vivo after transfusion is correlated with patient outcomes. However, from a molecular standpoint, promoting the longevity of cells has remained a challenge. In particular, improving the longevity of immune cells in vivo to fight disease is a major unsolved problem. Numerous embodiments of the present disclosure address this problem.

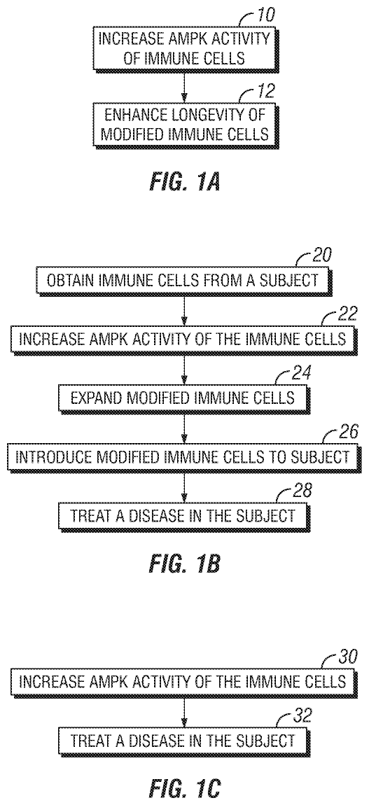

In some embodiments, the present disclosure pertains to methods of modifying immune cells. In some embodiments illustrated in FIG. 1A, the methods of the present disclosure include a step of increasing the AMP-activated protein kinase (AMPK) activity of the immune cells (step 10) to produce modified immune cells with enhanced longevity (step 12).

The methods of the present disclosure can have numerous embodiments. For instance, in some embodiments illustrated in FIG. 1B, the methods of the present disclosure include one or more of the following steps: obtaining immune cells from a subject (step 20); increasing the AMPK activity of the immune cells (step 22); expanding the modified immune cells (step 24); introducing the modified immune cells to a subject (step 26); and treating a disease in the subject (step 28). In further embodiments illustrated in FIG. 1C, the methods of the present disclosure include an in vivo method increasing the AMPK activity of immune cells in a subject (step 30) in order to treat a disease in the subject (step 32).

Additional embodiments of the present disclosure pertain to modified immune cells that have increased AMPK activity. The increased AMPK activity enhances the longevity of the modified immune cells.

As set forth in more detail herein, various methods may be utilized to increase the AMPK activity of various immune cells in order to enhance the longevity of the immune cells in various manners. Moreover, the modified immune cells of the present disclosure may be utilized in various manners to treat various diseases in various subjects.

Increasing AMPK Activity of Immune Cells

The AMPK activity of immune cells may be increased in various manners. For instance, in some embodiments, the increasing renders AMPK constitutively active in the immune cells.

The AMPK activity of immune cells may be increased in various environments. For instance, in some embodiments, the increasing occurs in vitro, such as in a petri dish. In some embodiments, the increasing occurs in vivo, such as in a subject.

Various in vitro and in vitro methods may be utilized to increase AMPK activity in immune cells. For instance, in some embodiments, the increasing occurs by introducing a gene that encodes a protein into the immune cells. In some embodiments, the introduced protein includes, without limitation, AMPK, Sirtuins, serine/threonine-protein kinase (STK11), CD36, trehalose transporter, derivatives thereof, fusion proteins thereof, subunits thereof, and combinations thereof.

In some embodiments, the introduced protein is AMPK. In some embodiments, the AMPK is the alpha subunit of AMPK, such as AMPK-α1, AMPK-α2, or combinations thereof. In some embodiments, the AMPK includes a mutation in the kinase active site that renders the expressed AMPK constitutively active.

In some embodiments, the introduced protein is a fusion protein that includes an oxygen dependent domain. For instance, in some embodiments, the fusion protein is AMPK, and the AMPK activity is regulated by the fused oxygen dependent domain. In some embodiments, the fusion protein is STK11, and the STK11 activity is regulated by the fused oxygen dependent domain. In some embodiments, the fusion protein is Sirtuin, and the Sirtuin activity is regulated by the fused oxygen dependent domain.

In some embodiments, the introduced protein becomes localized in the cytoplasm of the immune cells. In some embodiments, the introduced protein includes a mutation that localizes the protein in the cytoplasm, nucleus or the mitochondria of the immune cells. For instance, in some embodiments, the introduced protein is a mutated version of STK11, such that STK11 is localized in the cytoplasm of the immune cells by the mutation.

Proteins may be introduced into immune cells in various manners. For instance, in some embodiments, the introduction of proteins into immune cells includes introducing a gene that encodes a protein into the immune cells. In some embodiments, the introduction of proteins into immune cells includes introducing an expression vector into the immune cells. In some embodiments, the expression vector encodes and expresses the protein in the immune cells.

In some embodiments, the AMPK activity of immune cells is increased by exposing the immune cells to an activator. In some embodiments, the exposing occurs for at least 12 hours. In some embodiments, the exposing occurs for at least 24 hours. In some embodiments, the exposing occurs for at least 36 hours.

In some embodiments, the activator includes, without limitation, an AMPK activator, an STK11 activator, a Sirtuin activator, a CD36 activator, a Klotho activator, an autophagy activator, doxycycline, and combinations thereof.

In some embodiments, the activator is doxycycline. For instance, in some embodiments, STK11 activity is regulated by exposing the immune cells to doxycycline. In some embodiments, Sirtuin activity is regulated by exposing the immune cells to doxycycline. In some embodiments, CD36 activity is regulated by exposing the immune cells to doxycycline.

In some embodiments, the activator is an AMPK activator. In some embodiments, the AMPK activator includes, without limitation, sodium butyrate, trehalose, metformin, phenformin, 5-Aminoimidazole-4-carboxamide ribonucleotide (AICAR), aspirin, A-769662, resveratrol, MT 68-73, PF-06409577, PF-249, 5-(5-hydroxy-isoxazol-3-yl)-furan-2-phosphonic acid, and combinations thereof.

In some embodiments, the AMPK activity of immune cells is increased by inhibiting a protein. For instance, in some embodiments, the increasing includes the inhibition of Fyn.

Enhanced Longevity of Modified Immune Cells

The increasing of immune cell AMPK activity in accordance with the methods of the present disclosure can enhance the longevity of the modified immune cells of the present disclosure in various manners. In particular, the enhanced longevity of the modified immune cells of the present disclosure can be determined by various factors. For instance, in some embodiments, such factors include, without limitation, enhanced motility, persistent motility, enhanced cellular polarity, enhanced respiratory capacity, an enhanced number of punctate mitochondria, increased mitochondrial mass, reduced conjugation durations, enhanced spare respiratory capacity (SRC), enhanced capacity for oxidative metabolism, and combinations thereof. In some embodiments, the enhanced longevity of the modified immune cells of the present disclosure is determined by enhanced motility.

Immune Cells

The methods of the present disclosure can be utilized to enhance the longevity of various immune cells by increasing the AMPK activity of the immune cells. For instance, in some embodiments, the immune cells include human immune cells. In some embodiments, the immune cells include lymphocytes. In some embodiments, the lymphocytes include, without limitation, T-cells, NK-cells, B-cells, and combinations thereof.

In some embodiments, the lymphocytes include T-cells. In some embodiments, the T-cells include, without limitation, CD8⁺ T-cells, CD4⁺ T-cells, and combinations thereof.

In some embodiments, the immune cells include lymphocytes that have been engineered to express one or more immunoreceptors. In some embodiments, the immunoreceptors include chimeric antigen receptors (CAR) that target various receptors, such as CD19. In some embodiments, the engineered lymphocytes include T-cells, such as CAR T-cells.

In some embodiments, the lymphocytes are engineered to express one or more immunoreceptors after the AMPK activity is increased in the immune cells. In some embodiments, the immune cells are engineered to express one or more immunoreceptors before the AMPK activity is increased in the immune cells. In some embodiments, the immune cells are engineered to express one or more immunoreceptors at the same time that the AMPK activity is increased in the immune cells.

In some embodiments, the immune cells include lymphocytes that have been isolated from peripheral blood or from human tumors. In some embodiments, the immune cells include lymphocytes that have been engineered to express cytokines.

Obtaining Immune Cells from a Subject

In some embodiments, the methods of the present disclosure also include a step of obtaining immune cells from a subject. In some embodiments, the immune cells are obtained from a subject prior to increasing the AMPK activity of the immune cells.

Immune cells may be obtained from various sources in a subject. For instance, in some embodiments, the immune cells are obtained from a subject's peripheral blood. In some embodiments, the immune cells are obtained from a tumor in a subject.

Expanding Modified Immune Cells

In some embodiments, the methods of the present disclosure also include a step of expanding the modified immune cells of the present disclosure in vitro. For instance, in some embodiments, the modified immune cells of the present disclosure are expanded in vitro after the AMPK activity of the immune cells is increased.

Various methods may be utilized to expand the modified immune cells. For instance, in some embodiments, the modified immune cells are expanded by growing the modified immune cells in a petri dish.

Introduction of Modified Immune Cells to Subjects

In some embodiments, the methods of the present disclosure also include a step of introducing the modified immune cells of the present disclosure to a subject. Various methods may be utilized to introduce the modified immune cells of the present disclosure to a subject. For instance, in some embodiments, the introduction occurs by administering the modified immune cells of the present disclosure to a subject. In some embodiments, the administration occurs by a method that includes, without limitation, oral administration, inhalation, subcutaneous administration, intravenous administration, intra-nasal administration, intra-dermal administration, trans-dermal administration, intraperitoneal administration, intramuscular administration, intrathecal injection, topical administration, central administration, peripheral administration, transdermal administration, intraarterial administration, intracranial administration, intraspinal administration, intranasal administration, intraocular administration, intratumor administration, intramuscular administration, intranasal administration, subcutaneous administration, intra- or trans-dermal administration, intravenous administration, topical administration, or combinations thereof.

Increasing AMPK Activity of Immune Cells In Vivo

In some embodiments, the methods of the present disclosure can be utilized to increase the AMPK activity of immune cells in vivo in a subject. Methods of increasing the AMPK activity of immune cells in vivo in a subject were discussed previously. For instance, in some embodiments, the AMPK activity of immune cells is increased in vivo in a subject by introducing a gene (e.g., a gene on an expression vector) to the subject that encodes a protein in the immune cells (e.g., AMPK, Sirtuins, serine/threonine-protein kinase (STK11), CD36, trehalose transporter, derivatives thereof, fusion proteins thereof, subunits thereof, and combinations thereof). In some embodiments, the AMPK activity of immune cells is increased in vivo in a subject by introducing an activator to the subject (e.g., an AMPK activator, an STK11 activator, a Sirtuin activator, a CD36 activator, a Klotho activator, an autophagy activator, doxycycline, and combinations thereof). In some embodiments, the AMPK activity of immune cells is increased in vivo in a subject by inhibiting a protein in the subject (e.g., Fyn).

In additional embodiments, the AMPK activity of immune cells is increased in vivo in a subject by changing the diet of the subject. As such, in some embodiments, the methods of the present disclosure also include a step of changing the diet of the subject. In some embodiments, the changing of the diet occurs by restricting calories, fasting, or diet conditions that mimic fasting. In some embodiments, the changing of the diet occurs in a subject that is suffering from cancer. In some embodiments, the changing of the diet occurs in a subject that is suffering from cancer and undergoing cancer immunotherapy.

Utilization of Modified Immune Cells for Disease Treatment

The methods and modified immune cells of the present disclosure can be utilized to treat various diseases. As such, additional embodiments of the present disclosure pertain to utilizing the modified immune cells of the present disclosure to treat a disease in a subject.

For instance, in some embodiments, the disease to be treated by the modified immune cells of the present disclosure is a type of cancer. In some embodiments, the subject is a human being suffering from the cancer. In some embodiments, the subject is undergoing cancer immunotherapy.

In some embodiments, the cancer to be treated includes, without limitation, breast cancer, prostate cancer, pancreatic cancers, glioblastoma, acute lymphocytic leukemia (ALL), chronic myeloid leukemia (CML), lymphomas, leukemias, and combinations thereof. In some embodiments the cancer is breast cancer. In some embodiments the cancer is prostate cancer, pancreatic cancers, glioblastoma, ALL, CML, lymphomas or leukemias.

In some embodiments, the treatment first includes modifying immune cells in vitro in accordance with the methods of the present disclosure and then administering the modified immune cells to the subject. In some embodiments, the treatment also includes expanding the modified immune cells in vitro prior to administration. In some embodiments, the treatment includes modifying immune cells in vivo in accordance with the methods of the present disclosure.

Modified Immune Cells

Additional embodiments of the present disclosure pertain to the modified immune cells of the present disclosure. The modified immune cells of the present disclosure have an increased AMP-activated protein kinase (AMPK) activity that enhances the longevity of the modified immune cells.

In some embodiments, the modified immune cells of the present disclosure include an exogenous or endogenous AMPK that is constitutively active. In some embodiments, the modified immune cells of the present disclosure include an exogenous or endogenous AMPK that is over-expressed. In some embodiments, the AMPK includes, without limitation, the alpha subunit of AMPK, AMPK-α1, AMPK-α2, and combinations thereof.

In some embodiments, the modified immune cells of the present disclosure include lymphocytes. In some embodiments, the lymphocytes include, without limitation, T-cells, CD8⁺ T-cells, CD4⁺ T-cells, NK-cells, B-cells, and combinations thereof.

In some embodiments, the modified immune cells include lymphocytes that have been engineered to express one or more immunoreceptors, such as chimeric antigen receptors (CAR). In some embodiments, the modified immune cells include CAR T-cells.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. T-Cell Potential for CD19-Expressing Malignancies Revealed by Multi-Dimensional Single-Cell Profiling

T-cell therapy with specificity redirected through chimeric antigen receptors (CARs) has shown efficacy for the treatment of hematologic malignancies. However, understanding why some genetically modified T cells have improved fitness confounds manufacturing and predicting therapeutic success. Millions to billions of patient-derived T cells are manufactured and infused, which requires new approaches to deconvolute the heterogeneity of T-cell populations and map the myriad of T-cell functions and phenotypes.

In this Example, Applicant utilized a suite of technologies based on high throughput at single-cell resolution based upon Timelapse Imaging Microscopy In Nanowell Grids (TIMING) that integrates cytokine profiling, mapping transcriptome, and profiling energetics to show that persistent motility and matched cell polarity of CD19-specific CAR⁺ T cells is the defining feature that predicts desired polyfunctionality resulting in anti-cancer effects. While molecular profiling and confocal microscopy identified CD2/CD58 axis as a marker of T cells with high persistent motility, Applicant employed a marker-free sorting strategy for enriching T cells with persistent motility and validated the in vivo anti-leukemia efficacy of motile T cells. In aggregate, Applicant's integrated single-cell data identified that independent of CAR design or biomanufacturing, persistent motility serves as a selectable cell-intrinsic biomarker, desired in the bioactivity of expanded CAR⁺ T cells.

T cells traffic to sites of disease, and the ability to migrate to and within all tissues within the human body differentiates effective T-cell therapeutics from other targeted immunotherapies based on the infusion of recombinant proteins. Few tools are available to examine the migratory properties of T cells, despite this being a central feature of their immunobiology with the inherent benefit that they can target to tumor deposits, recycle effector functions within malignancies, and persevere through cancer lesions despite elevated interstitial pressures.

The administration of immune effector cells propagated ex vivo have been shown to be effective for the treatment of solid cancers such as melanomas and liquid tumors such as acute and chronic B-cell leukemias. T cells stably endowed with a genetically encoded chimeric antigen receptor (CAR) targeting CD19 have shown remarkable clinical responses in patients' B-lineage leukemias and lymphomas refractory to other treatments. The potential for complete responses has spurred the development of CARs targeting other antigens for the treatment of hematologic malignancies and invasive cancers. The field of CAR T cells has exploded with the culmination of the food and drug administration (FDA) approval of Kymriah and Yescarta, and while attention has been devoted to antigen discovery and CAR design, identifying metrics that define the functional potential and thus the therapeutic potential of T-cell products is limited.

Because of inter- and intra-tumor heterogeneity, technologies that aggregate T-cell biology are unable to accurately capture the complexities of a T-cell product with defined and desired characteristics. For example, it is widely accepted that less differentiated cells (central memory [T_{CM}] or stem memory [T_{SCM}] T cells) have increased proliferative capacity and thus sustained persistence. However, it is emphasized that the multitude of individual cells vary in their persistence and functional potential.

Although the persistence of infused T cells correlates with the efficacy of tumor eradication in leukemias, recent pre-clinical data suggest that beyond tumor infiltration and sustained engraftment, the ability of these cells to recycle effector functions within the tumor microenvironment (TME) is an essential attribute for tumor eradication. The implementation of biomarkers that adequately define the therapeutic potential of T cells at the single-cell level, integrated over the entire cell population, is vital for the standardization of T-cell based therapies.

Here, Applicant employs a suite of integrated single-cell technologies, including timelapse imaging microscopy in nanowell grids (TIMING), cytokine profiling, transcriptional, and mitochondrial profiling to explore the dynamic behavior of individual T cells. Applicant's results profiling thousands of cells demonstrate that T-cell polyfunctionality is correlated with persistent T-cell motility and cellular polarity.

In order to gain mechanistic insight into the link between polyfunctionality and persistent motility, Applicant performed comparative multiplexed single-cell transcriptional profiling on T cells with persistent motility (motile) and non-motile cells. These results revealed that interaction between CD2 and CD58 is associated with polyfunctionality. To take advantage of movement as a desired T-cell trait, Applicant implemented a sorting technology based on 3D motility to enrich for T cells with persistent motility. The motile T cells were more naïve like with elevated respiratory capacity, and an increased number of punctate mitochondria (indicative of dynamic mitochondria).

At the molecular level, Applicant demonstrates that the metabolic regulator AMP kinase (AMPK) is required for persistent T-cell motility. The optimal properties of these motile T cells were confirmed in a mouse model of leukemia wherein the T cells persisted longer leading to more sustained responses.

In aggregate, Applicant's data obtained by integrating function, phenotype, transcriptional profiling, and metabolism, demonstrate that persistent motility is a selectable cell-intrinsic biomarker reflective of the bioactivity of expanded T cells.

### Example 1.1. Killer CAR⁺ T Cells Detach Faster from Target Cells in Comparison to IFN-γ Secreting Cells

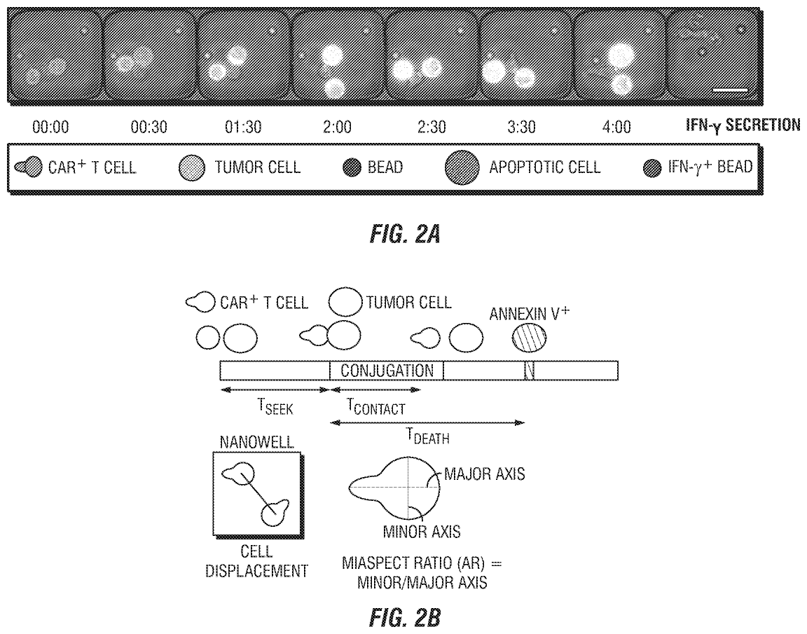

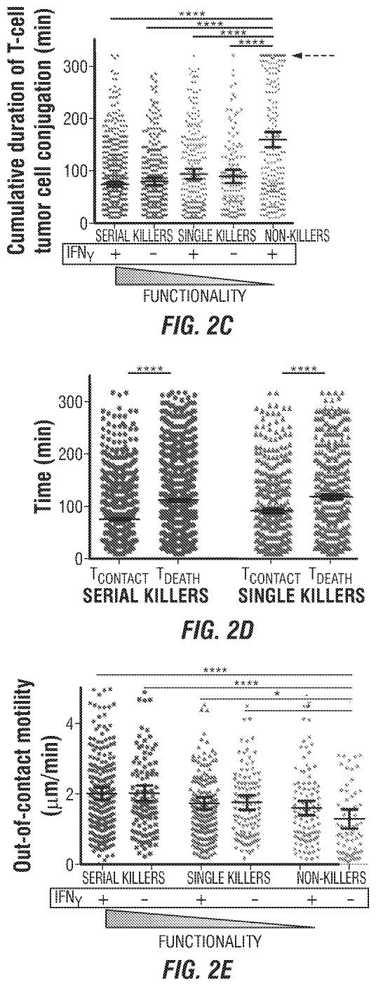

Applicant utilized the TIMING platform to profile the polyfunctionality of T cells and link it to their molecular profiles. CD19-specific CAR⁺ (designated 19-28z) human T cells comprised of almost exclusively of CD8⁺ T cells were generated, and their antigen specificity was confirmed by measuring IFN-γ production. CAR⁺ T cells as effectors, NALM-6 tumor cells as targets, and pre-functionalized beads coated with IFN-γ capture antibody as cytokine sensors, were loaded sequentially onto a nanowell grid array, and the kinetics of killing and end-point cytokine secretion from the same cells was monitored using TIMING (FIG. 2A).

Applicant identified 1,178 nanowells of interest containing a single live T cell, 2 to 5 tumor cells, and one or more beads. Since every T cell within this subset was incubated with multiple tumor cells, three functional definitions were utilized: serial killer cells that killed at least two tumor cells, single killer cells that killed exactly one tumor cell, and monofunctional cells that did not kill but only secreted IFN-7.

To define the kinetics of interaction between individual T cells and tumor cells that lead to subsequent killing, two interaction parameters, t_{Contact}, cumulative duration of conjugation between the first contact to target death; and t_{Death}, the time between first contact and target apoptosis, were computed (FIG. 2B). Applicant observed that T cells that only secreted IFN-γ (monofunctional) exhibited the longest conjugation durations of all functional (killing and/or IFN-y secretion) T cells. This duration was significantly longer than cells that killed either only one tumor cell with or without IFN-γ secretion, or multiple tumor cells with or without IFN-γ (FIG. 2C). These differences were dominated by the presence of a subpopulation of T cells within the IFN-γ monofunctional cells that remained conjugated to the tumor cell for the entire period of observation (FIG. 2C).

For both single-killers and serial killers, t_{Contact} was significantly lower than t_{Death}, demonstrating that T cell detachment preceded tumor-cell showed Annexin V staining (FIG. 2D). Additionally, the total duration of conjugation of all killer T cells was lower than non-killer T cells, suggesting that the termination of the conjugation is rapid in killer T cells.

Applicant next investigated if intrinsic T-cell properties such as persistent motility prior to tumor-cell conjugation might offer insights into their functional capacity subsequent to tumor cell conjugation. Individual 19-28z T cells that displayed cytotoxicity upon tumor cell conjugation had higher out-of-contact motility than T cells that did not (FIG. 2E). Across the different functional subsets of T cells, there was an association between decreasing out-of-contact motility and lowering poly-functionality. Consistent with this observation, except for monofunctional T cells that only secreted IFN-γ, all other populations demonstrated significant differences in motility in comparison to the non-functional population (FIG. 2E).

These observations were also consistent when measuring motility during conjugation with the tumor cell, where polyfunctional cells and specifically serial killers had higher motility in comparison to the non-functional cells. These results suggest that polyfunctional T cells benefit from high motility, which could reflect an elevated basal level of activation that allows rapid cytolytic synapse formation, or a facilitated discovery of tumor cells within the local microenvironment, and subsequent lysis of target cells.

### Example 1.2. Persistent T-Cell Motility Enables Selection of Functional T Cells

Applicant sought to utilize persistent motility as a selectable property for T-cell bioactivity. Applicant hypothesized that T cells with persistent motility could be enriched using a modified transwell assay, with pore sizes consistent with previously published confinement studies of T cells in vitro. Applicant initially tested the effect of functionalization of the membrane by coating with fibronectin or collagen, but in comparison with the uncoated membrane, these did not significantly increase the number of migrated (motile) cells (not shown).

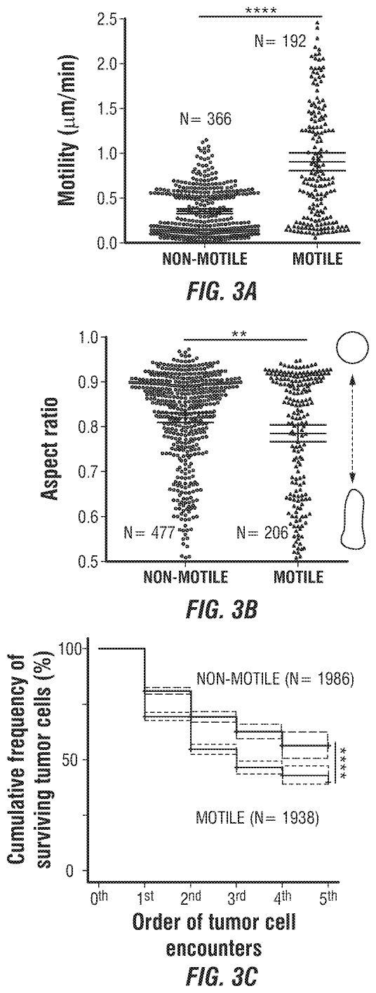

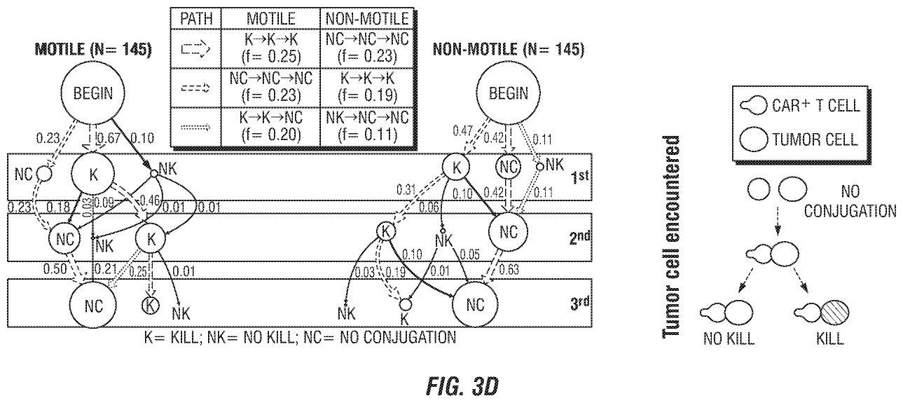

Although all the data described to this point were obtained from CD8⁺19-28z T cells, Applicant aimed to ensure that this effect was generalizable to all T cells, both CD4 and CD8, and hence Applicant used populations of 19-28z T cells that comprised a mixture of both phenotypes. Unstimulated 19-28z T cells were seeded onto a Boyden chamber, and motile T cells were harvested from the bottom chamber and compared to cells from the top chamber (non-motile) or unsorted cells. TIMING assays tracking single-cell motility confirmed that the cells harvested from the bottom chamber had increased persistent motility and polarization in comparison to the cells harvested from the top chamber, validating the transwell assay (FIGS. 3A-B).

To dissect the cytotoxic capacity at the single-cell level, Applicant performed TIMING assays with live motile or non-motile 19-28z T cells as the effector cells and NALM-6 tumor cells as targets. Consistent with Applicant's previous TIMING data (FIG. 2), motile T cells participated in killing and serial killing at significantly higher frequencies in comparison to the non-motile population (FIG. 3C). Since Applicant recognized that the TIMING data is gated based on live T cells that conjugate to tumor cells (i.e. ones that establish a synapse), and posited that migration is likely to impact the ability of T cells to seek and conjugate to tumor cells, Applicant modeled the time evolution of the interaction and outcomes of the interaction between individual T cells and tumor cells using a state transition diagram.

Consistent with Applicant's hypothesis, the most probable path for motile T cells was to encounter multiple tumor cells and to behave as serial killers whereas non-motile T cells were characterized by an impediment in conjugating to tumor cells (FIG. 3D). Collectively, these results suggest that the motile T cells isolated by the transwell assay were persistent serial killers.

### Example 1.3. Motile T Cells have Signatures of Less Differentiated Cells and have a High Spare Respiratory Capacity (SRC)

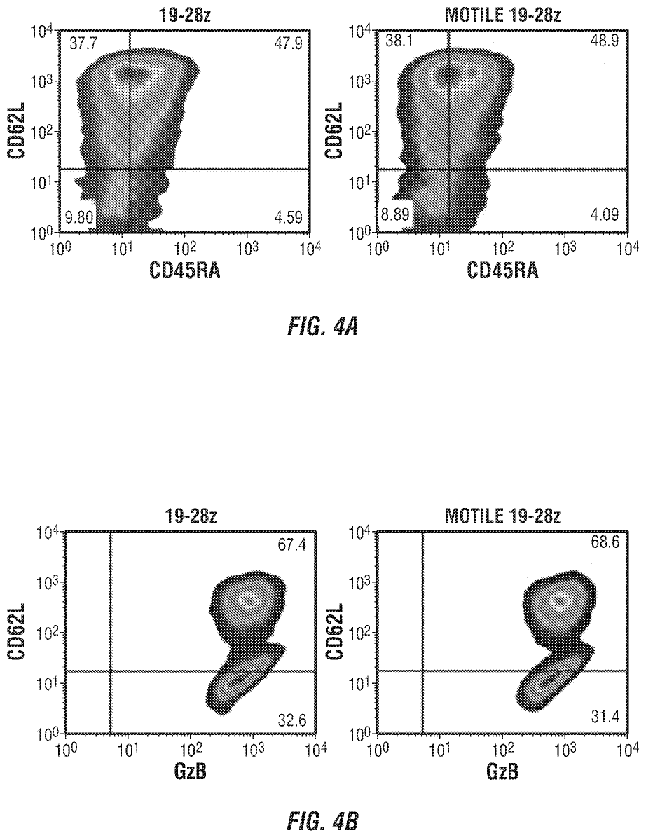

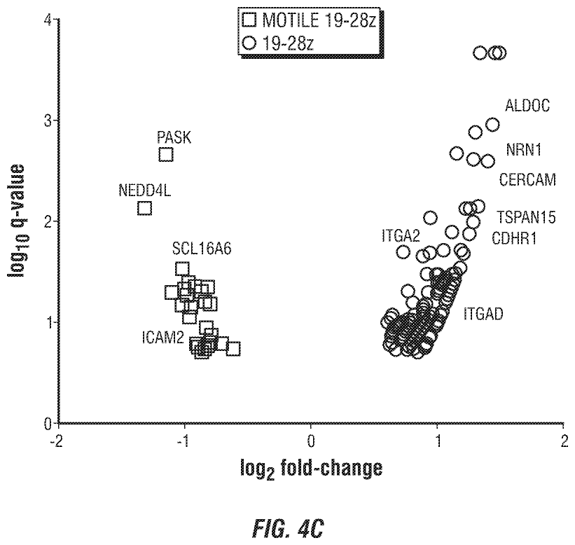

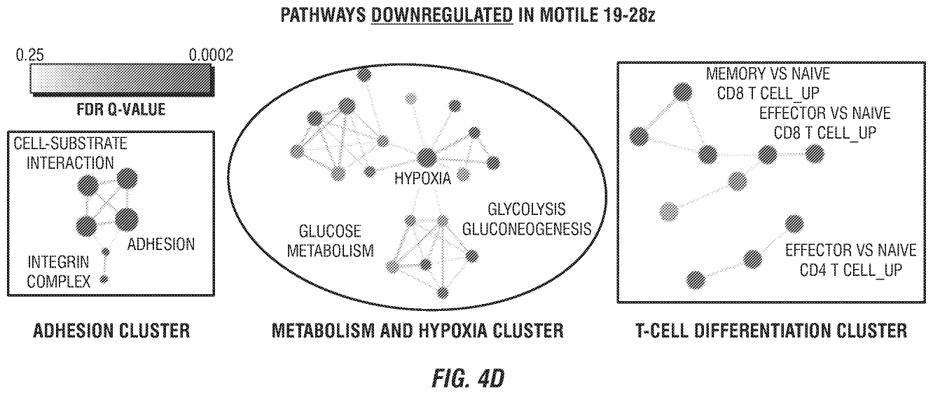

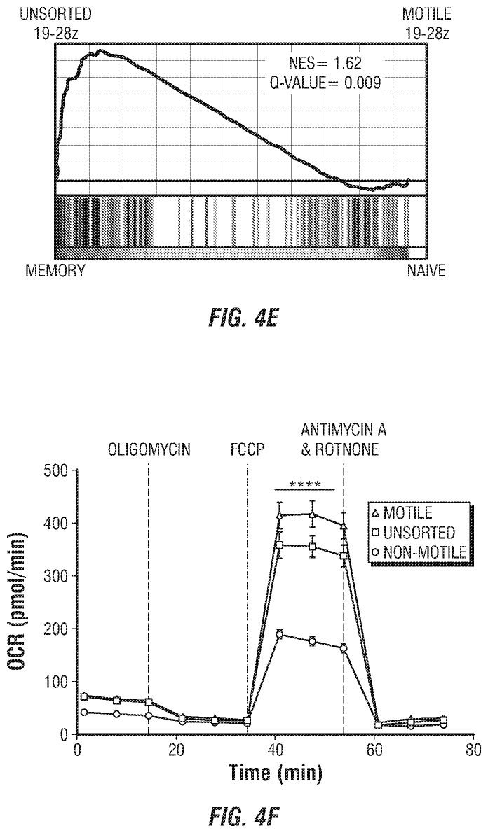

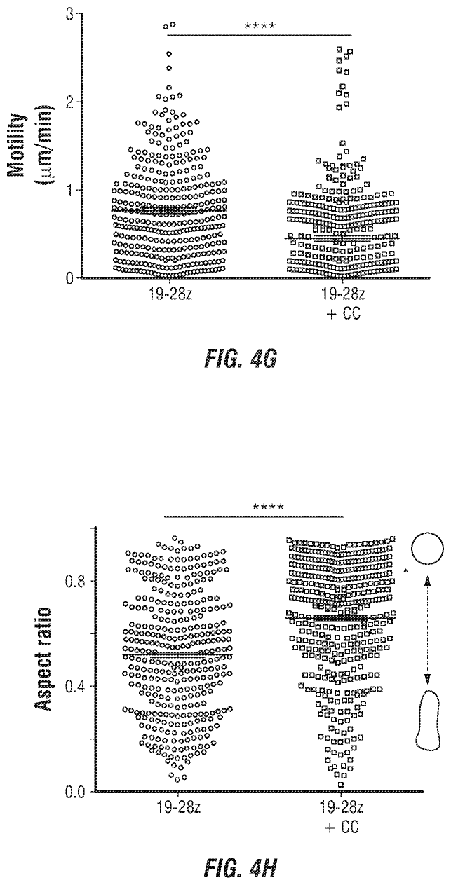

Next, Applicant aimed to determine if the sorted motile 19-28z T cells had properties that distinguished the motile population from the parent, unsorted population. To test if the differences in the motility and functional properties of motile T cells could be explained and reflected by their phenotype, Applicant compared the memory phenotype of motile and of unsorted T cells, and observed that both populations were comprised predominantly of naïve (CD62L⁺CD45RA⁺) and central memory (CD62L^{neg}CD45RA⁺) CAR⁺ T cells (FIG. 4A). Both motile and unsorted populations had no difference in expression of either the CAR or intracellular Granzyme B (FIG. 4B), which was consistent with transcriptional and functional data above.

Although Applicant had performed single-cell transcriptional profiling on motile cells, this was performed using a targeted panel of preselected genes. In order to gain a comprehensive understanding of the differences between motile and unsorted cell populations, Applicant performed RNA-seq. Principal component analysis (PCA) confirmed that the differentially-expressed genes (DEGs) could clearly segregate the two distinct populations. A total of 144 DEGs were identified, and transcripts related to cellular adhesion were downregulated in motile 19-28z T cells in comparison to unsorted 19-28z T cells (FIG. 4C). Analyses of pathways related to cellular migration confirmed that pathways related to focal adhesion, cell-ECM interactions, and integrin complexes were downregulated in the motile 19-28z T cells (FIG. 4D).

A separate cluster of pathways related to metabolism showed that motile 19-28z T cells had lower expression of genes associated with glycolysis and hypoxia compared to the unsorted 19-28z T-cell populations (FIG. 4D). Gene-set enrichment analyses (GSEA) with the Molecular Signatures Database specific to T cell datasets revealed that the motile 19-28z T cells had a less differentiated phenotype in comparison to the unsorted 19-28z T cells (FIGS. 4D-E). Collectively, whole transcriptome profiling suggested that the motile 19-28z T-cell populations were comprised of naïve-like T cells.

The bioenergetic and metabolic needs are linked to the differentiation status of T cells. To further advance the bioenergetic differences identified by RNA-seq, Applicant measured the oxygen consumption rate (OCR) of both the motile and unsorted T-cell populations. Metabolic flux analyses revealed that the motile populations had both higher maximal respiratory capacity and spare respiratory capacity (SRC) in comparison with either the unsorted or non-motile T-cell populations (FIG. 4F).

Single-cell confocal microscopy also demonstrated that motile 19-28z T cells had increased mitochondrial mass and an increased number of punctate mitochondria (suggestive of fission) in comparison to the non-motile 19-28z T cells. Since AMP-kinase (AMPK) is a well-known regulator of mitochondrial mass and integrity, Applicant next sought to determine if the kinase activity of activated AMPK (phosphorylation in the a-subunit) was the molecular link between persistent motility and metabolism that Applicant documented.

Accordingly, Applicant inhibited the activity of AMPK in 19-28z T cells using the small molecule inhibitor dorsomorphin (compound C, CC). Cells treated with CC showed profound defects in morphology and persistent motility, confirming that AMPK activity is essential for the motility of T cells. (FIGS. 4I-J). Consistent with impaired motility, CC treated 19-28z T cells showed a decreased propensity to conjugate to tumor cells in single cell assays, in comparison to DMSO-treated 19-28z T cells.

Furthermore, treated 19-28z T cells that did make contact with target cells showed significantly longer conjugation times and delayed induction of tumor cell apoptosis when compared to the untreated 19-28z T cells. Applicant also confirmed that this requirement of AMPK for persistent motility was generalizable to genetically unmodified, tumor-reactive T cells and not just CAR⁺ T cells. Collectively, these results show that motile 19-28z T cells have a greater capacity for oxidative metabolism in comparison to the unsorted or non-motile 19-28z T cells, and that the activation of the metabolic regulator, AMPK, is essential for T cell motility.

### Example 1.4. Motile T Cells Reject Established Leukemia and Sustain Persistence In Vivo

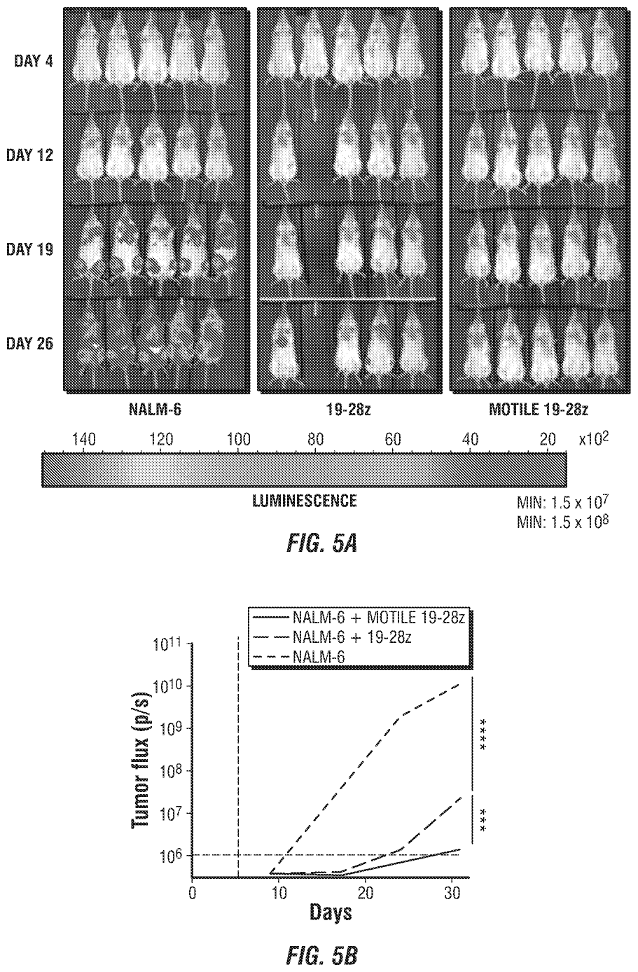

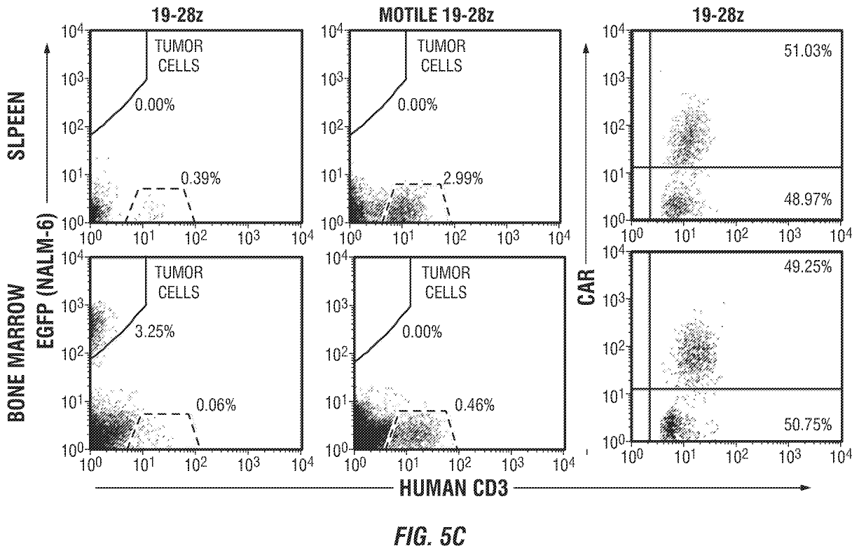

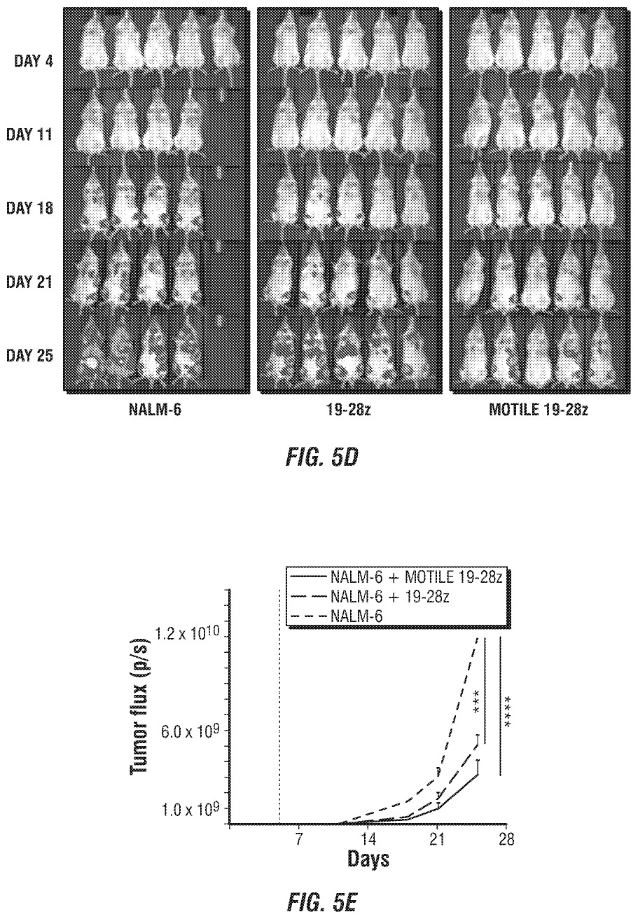

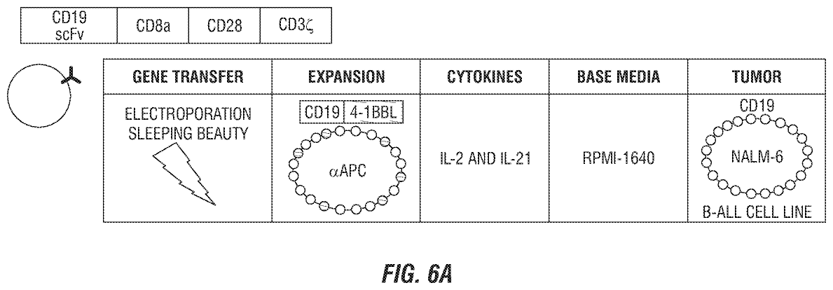

Based on the in vitro functional, transcriptional imaging, and bioenergetics data, Applicant hypothesizes that motile T cells should be able to promote CAR⁺ T-cell persistence in vivo leading to improved antitumor efficacy. Applicant assessed the efficacy of the motile 19-28z T cell population using a model of established leukemia. NOD.Cg-Prkdcscid Il2rgtm1Wjl/SzJ (NSG) mice were injected with CD19⁺ NALM-6 human leukemia cells transduced with firefly luciferase, and 5 days after tumor-cell engraftment, were treated with 19-28z CAR⁺ T cells. The motile 19-28z T cells demonstrated potent and superior anti-tumor activity, reducing tumor burden to the detection limit, with tumor flux significantly lower in comparison to the unsorted 19-28z T cells (FIGS. 5A-B). It is worth emphasizing that this improvement in efficacy was obtained purely by isolation of the subpopulation of motile cells in the population without any additional culturing or modifications. In both the bone marrow (BM) and spleen, mice treated with motile 19-28z T cells harbored no tumor cells, but only persisting CAR⁺ T cells (FIG. 5C).

By contrast, Applicant could detect the outgrowth of tumor cells within the BM of mice treated with unsorted 19-28z T cells, which was accompanied by a very low frequency of CAR⁺ T cells in the BM in these same mice (FIG. 5C). Similarly, while Applicant could detect CAR⁺ T cell persistence in the spleen of mice infused with motile 19-28z T cells, there were very low frequencies of CAR⁺ T cells in the spleen of the mice that received the unsorted 19-28z T cells (FIG. 5C).

In an independent experiment, Applicant compared the efficacy of motile and unsorted 19-28z T cells at suboptimal doses against these same NALM-6 tumors in vivo. Motile 19-28z T cells showed significantly enhanced anti-tumor activity compared to the unsorted 19-28z T cells (FIG. 5D). In aggregate, these results demonstrate that motile 19-28z T cells traffic efficiently to the BM and spleen, and display enhanced persistence and prolonged tumor control, even at suboptimal doses.

### Example 1.5. Motility is a Conserved Biomarker of Functional T Cells Independent of CAR Design or Biomanufacturing Protocols

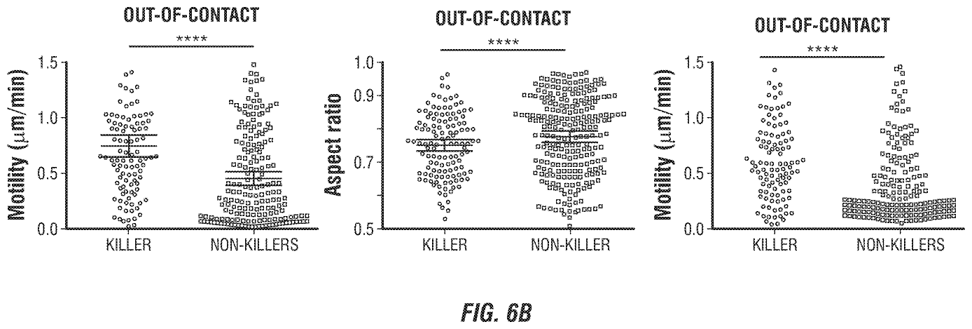

Since all of Applicant's results were obtained with a single CAR design and manufacturing protocol, Applicant tested if the link between motility and functionality is generalizable to other CD19 derived CARs. Accordingly, Applicant utilized TIMING to first compare the persistent motility of killer and non-killer CAR⁺ T cells that were engineered with a CD19-specific CAR containing the CD8 hinge and transmembrane regions (19-8-28z) (FIG. 6A). Consistent with Applicant's other data, individual killer 19-8-28z T cells demonstrated higher persistent motility both with and without conjugation to NALM-6 tumor cells (FIG. 6B).

### Example 1.6. Discussion

With the recent federal regulatory approval of CD19-specific CAR⁺ T cells as living drugs, there is a need to define biomarkers that provide insights into the potential clinical impact of adoptive immunotherapy. Among the different T-cell effector functions that have been characterized, persistent T-cell motility has not been characterized in the context of numerically expanded T cells. Preclinical studies of adoptive cell transfer (ACT) using two-photon microscopy have clearly demonstrated that mouse T-cell motility prior to engagement and killing of tumor cells is an essential component of their efficacy. Persistent T-cell motility enables these cells to search for the cognate target cells, and this exploration is an essential pre-requisite for catalyzing any anti-tumor functionality. For the efficacy of T-cell based therapies in facilitating lasting remissions, it is important to enable random exploration through many different compartments and not just targeted homing to pre-defined tissue (e.g., through the enforcement of defined chemokine receptors).

Applicant's results of interrogating the polyfunctionality of 19-28z CAR⁺ T cells demonstrate that at the single-cell level, non-killer, IFN-γ secreting cells have extended periods of conjugation. The existence of a subpopulation of CAR⁺ T cells with extended conjugation leading to chronic IFN-γ secretion suggests a new hypothesis for CAR engineering. Since the affinity of the CAR is the primary determinant of the duration of conjugation between the T cell and the tumor cell, tuning the affinity of the CAR molecule by engineering the off rate should allow for CARs with preserved polyfunctionality but without chronic IFN-γ secretion. It is worth exploring if CARs engineered with this paradigm might show efficacy without the toxicity associated with chronic IFN-γ secretion.

The integrated transcriptional, phenotypic and functional single-cell data demonstrated an association between CD2 (LFA-2) and CD58 (LFA-3), and persistent motility. Although the significance of CD2 is highlighted from pan-cancer studies, it was inferred to merely reflect the presence of infiltrating lymphocytes. Applicant's combined functional, transcriptional and phenotypic data advances the role of CD2-CD58 interactions at the single-cell level, and is consistent with independent studies probing the genes/proteins essential for cancer immunotherapy using CRISPR-Cas9 screens that mimic loss-of-function mutations involved in resistance to these therapies.

Similarly, it has been previously shown that CD2 costimulation can drive T cells to a non-exhausted state and prevent the induction of inhibitory receptors like PD-1. Thus, CAR structures that incorporate CD2 endodomains may be worth comparing to the standard CD28 and CD137 endodomains.

From a clinical perspective, it is well-documented that mutations in CD58, leading to loss of function, are prevalent in diffuse large B-cell lymphoma, and that these mutations might be associated with resistance or relapse. As the number of patients being treated by CD19-specific CAR⁺ T cells expands, it will be important to monitor if escape from T-cell treatment is also accompanied by the expansion of CD58 negative tumor cells, and whether CAR⁺ T cells harboring a CD2 endodomain can mitigate this escape.

It has been documented that naïve T cells, both in vivo and in vitro, display features of Brownian motion and that their ability to scan lymph nodes efficiently can be independent of integrins. Applicant's selection relied on persistent motility guided by 3D confinement, and the RNA-seq data demonstrated that the motile T cells had decreased expression of the adhesion molecules, and were less differentiated cells. These observations are consistent with the model that motile cells are naïve like cells in both molecular signature and migratory properties.

T cells with high persistent motility also have a high bioenergetic requirement to support the locomotion of these cells. Applicant's results confirmed that the motile CAR⁺ T cells had enhanced mitochondrial SRC, a property that likely promotes long-term survival, and this is consistent with previous reports that demonstrated that naïve CAR⁺ T cells have enhanced SRC.

3D confocal microscopy also demonstrated increased punctate mitochondria in individual cells with persistent motility. Within this context, since AMPK is a well-known master regulator of SRC and mitochondrial remodeling, Applicant confirmed that inhibiting AMPK has a profound impact on persistent T-cell motility and consequently function at the single-cell level. To the best of Applicant's knowledge, this is the first direct demonstration of the role of AMPK in persistent T-cell motility at the single-cell level, and this sets the stage for additional studies that explore this link between AMPK as a modulator of energetics and function within individual T cells and their anti-tumor efficacy.

Applicant's data is also consistent with studies that have demonstrated activated AMPK as a negative regulator of cellular adhesion and integrin activity in other cell types. However, it is also important to recognize that AMPK is a global modulator of many different aspects of cell biology that need to be mapped within the context of T cells.

Collectively, Applicant's results in this Example portray the motile 19-28z T cells as having increased bioactivity and functionality leading to persistence in vivo and control of tumor growth. However, Applicant recognizes that, while the persistent motility of these CAR⁺ T cells is a selectable property and reflective of the bioactivity of cells within a given population, it is unknown whether persistent motility by itself can be utilized as a comparative marker in evaluating CAR⁺ T cell populations with varied CAR designs. In this regard, Applicant's own data comparing 19-28z T cells and 19-8-28z T cells manufactured from the same donors illustrates that 19-8-28z T cells have higher persistent motility than 19-28z T cells when evaluated at the single-cell level using TIMING, and this is consistent with their in vivo bioactivity.

Moreover, while Applicant has demonstrated the implementation of a simple transwell assay for the enrichment of motile 19-28z T cells, as the data demonstrates, the segregation of cellular populations is not perfect. For translational purposes, Applicant anticipates having to build custom microfluidic chips capable of efficient segregation of large numbers of motile T cells.

Additionally, Applicant recognizes that persistent motility might not necessarily be imprinted on T cells and their daughter cells, as they undergo cell division in vivo. Nonetheless, persistent motility enables the identification and isolation segregation of T cells in vitro with long-lived potential in vivo.

In some embodiments, identification of subsets of T cells with improved therapeutic potential can be used to refine manufacturing and improve anti-tumor effects. Applicant identified that persistent motility is a marker that selects for T cells with superior anti-tumor effects that is reflective of productively activated cells that have the balanced ability for functional execution without compromising proliferative potential. Applicant anticipates that the ability to track T-cell potency by monitoring persistent motility in the presence and absence of cognate tumor cells will enable the more systematic manufacturing of more potent T cells.

### Example 1.7. Cell Lines and Primary T Cells

Human pre-B cell leukemic line NALM-6 (ATCC) were cultured in T-cell medium (RPMI+10% FBS) and used as CD19⁺ target cells. A second-generation CAR signaling via CD28 and CD3-ζ endodomains (with an IgG4 spacer) were expressed in human T cells by electroporation with DNA plasmids from the Sleeping Beauty (SB) transposon/transposase system, as described previously³⁵. T cells were used between 14 days and 28 days after transfection.

### Example 1.8. Beads Preparation: Coating Beads with the Primary Capture Antibody

One L of Promag 3 Series goat anti-mouse IgG-Fc beads (˜2.3×10⁵ beads) in solution was washed with 10 μL of PBS and re-suspended in 19.6 μL PBS (˜0.05% solids). Mouse anti-human IFN-γ (clone 1-D1K) was then added to beads at a final concentration of 10 μg/mL and incubated for 30 min at room temperature, followed by washing and re-suspension in 100 μL PBS.

### Example 1.9. Nanowell Array Fabrication and Cell Preparation

Nanowell array fabrication for interrogation of effector functions at single-cell level was performed as described previously. Approximately 1 million effector cells and target cells were both spun down at 400×g for 5 min followed by labeling with 1 μM PKH67 and PKH26 fluorescent dyes respectively according to the manufacturer's protocol. Excess unbound dyes were then washed away and cells were re-suspended at ˜2 million cells/mL concentration in complete cell-culture media (RPMI+10% FBS).

### Example 1.10. TIMING Assays for the Multiplex Study of Effector Cytolytic Phenotypes and IFN-γ Secretion

Capture antibody coated beads and labeled effector and target cells were loaded consecutively onto nanowell arrays. Whenever necessary, arrays were washed with 500 μL of cell culture media to remove excess beads or cells. Next, detection solution containing Annexin V-Alexa Fluor 647 (AF647) (Life Technologies) (for detection of target apoptosis) was prepared by adding 50 μL of stock solution to 2.5 mL of complete cell-culture media without phenol red. Nanowell arrays were then imaged for 6 hours at an interval of 5 minutes using LEICA/ZEISS fluorescent microscope utilizing a 20×0.80 NA objectives and a scientific CMOS camera (Orca Flash 4.0 v2). At the end of the timelapse acquisition, biotinylated mouse anti-human IFN-γ antibody was added to 2.5 mL cell media above at 1:1000 dilution. This was incubated for 30 minutes followed by washing and incubation with 5 μg/mL Streptavidin-R-Phycoerythrin (PE). The entire chip was again imaged to determine the intensity of PE signal on the microbeads and the two datasets were matched using custom informatics algorithms.

### Example 1.11. Image Processing, Cell Segmentation, Cell Tracking, and Data Analytics

Image analysis and cell segmentation/tracking were performed as described previously. The pipeline of image processing and cell segmentation ends with statistical data analysis based on the tabular spatiotemporal measurement data generated by the automated segmentation and cell tracking algorithms. Nanowells containing 1 effector and 2-5 tumor cells were selected for further analysis. Applicant then partitioned all these events based on the functionalities of the cells (i.e., mono-kill, serial kill, and IFN-γ secretions). A size-exclusion filter based on maximum pixel areas were used to effectively differentiate cells from beads (beads were much smaller than cells). Where specified, cell tracks were represented using MATLAB (Mathworks Inc. MA).

For tracking the mitochondria within the cells using confocal microscopy, Z-stacks of 16-bit images were extracted for each channel and processed in ImageJ using a series of plugins. First, the Subtract Background plugin was applied on the mitochondria channel prior to segmentation to reduce variations in background intensities. Next, the 3D Objects Counter plugin was applied to the background-corrected image to determine mitochondrial ROIs. These ROIs were overlaid onto the original image and measurements were collected. Similarly, the 3D Objects Counter plugin was also used on nuclei but using the original image only. Lastly, tracking of single cell movement was done using the TrackMate plugin in order to filter out unstable cells upon their movement. All measurements were consolidated in R, where mitochondria and nuclei were matched to their corresponding cell.

### Example 1.12. Gene Expression Profiling

PKH67 stained CD8⁺ T cells were loaded on a nanowell array, immersed with Annexin V-AF647 containing phenol red-free complete cell-culture medium and imaged for 3 hours using TIMING exactly as described above. After carefully washing the cells on the chip 3 times with cold PBS (4° C.), cells were kept at 4° C. until retrieval. Time-lapse sequences were manually analyzed to identify live high and low motility cells. The cells were individually collected using an automated micro-manipulating system (CellCelector, ALS) and deposited in nuclease-free microtubes containing 5 μL of 2× CellsDirect buffer and RNase Inhibitor (Invitrogen). Single cell RT-qPCR was then performed using the protocol ADP41 developed by Fluidigm. Ninety-two cells (48 motile and 44 non-motile) were assayed, along with bulk samples of 10 and 100 cells, and with no-cell and no-RT controls. The panel of 95 genes included genes relevant to T cell activation, signaling and gene regulation, and was designed and manufactured by Fluidigm D3 AssayDesign.

For data analysis, Applicant first extracted Log 2Ex value by subtracting Ct values from a threshold of 29, as described previously. Applicant then excluded data from i) cells that had less than 40% of genes that were amplified and had a mean of Log 2Ex out of the range of population mean±3SD and from ii) genes that were amplified in <10% of cells. Post-process analysis was done using Excel (Microsoft), Prism (GraphPad), MeV⁴⁰, STrenD⁴¹ and Genemania webtool (http://www.genemania.org/).

### Example 1.13. Migration of T Cells Through a Transwell Migration Chamber

Unstimulated, overnight serum deprived, CAR⁺ T cells were seeded on the top compartment of PET 5 or 8 m-pore Boyden transmigration chamber (EMD Millipore), while the lower compartment contained FBS rich media. After 4-6 hours, the cells from the bottom and the top compartment were harvested as “motile” and “non-motile” populations, respectively. The lower part of the membrane is washed into the “motile” cell suspension, while the top surface of the membrane is washed into the “non-motile” cell suspension. Cells are analyzed for phenotype and function using flow cytometry and TIMING.

### Example 1.14. Flow Cytometry-Based Phenotyping, Cytokine Secretion, and Cytotoxicity Assay

For phenotyping, CAR⁺ T cells were stained using a panel of human-specific antibodies CD107a (H4A3), CD2 (RPA-2.10), CD58 (1C3), CD244 (2-69), CD62L (DREG-56), CD45RA (HI100), CD45RO (UCHL1), CD95 (DX2), CD3 (SK7), CD27 (L128, MT271), CD28 (L293), CD25 (M-A251), CD127 (HIL-7R-M21), KLRG1 (2F1/KLRG1) CD57 (NK-1). CD4 (OKT4), CD8 (RPA-T8), CD69 (FN50), and CCR7 (G043H7) were from Biolegend. The anti-CAR scFv was made in house. To assay cytotoxicity of the cells at the population level, NALM-6 target cells were stained with PKH26, and co-culture with T cells were set in triplicate at different T cell: target cell ratios, with 100,000 T cells per well. Fluorescently labeled anti-CD107a antibodies together with GolgiStop (BD Biosciences) at 0.7 μL/mL was added to the co-culture to stain for degranulating cells. After the assay, cells were washed and stained with Zombie Aqua, antibodies against CD4, CD8, CD3, and CD69, then analyzed by flow cytometry. To measure cytokine release in the supernatants of the co-cultures, IFN-γ and TNF-α were quantified using the MultiCyt® QBeads™ Human PlexScreen (Intellicyt) following the manufacturer's protocol. For the blocking experiments, CAR⁺ T cells were incubated for 24 hours in flat bottom 96 well plates that were pre-coated overnight with 10 μg/mL of purified anti-CD244 (clone C1.7, Biolegend), CD2 (clone LT2, Miltenyi) or anti-CD58 (1C3, BD Biosciences) and rinsed once with complete culture medium before performing the functional assay.

### Example 1.15. TIMING and Confocal Microscopy

CAR⁺ T cells were seeded onto nanowell arrays and the motility of the cells monitored using TIMING. At the end of two hours, the cells on the chip were washed carefully three times with cold PBS-5% FBS (4° C.). Fluorescently conjugated monoclonal antibodies against CD2, CD244 or CD58 were added to the chip, incubated for 30 min at 4° C., washed and imaged on a Nikon Ellipse TE confocal microscope fitted with a 60×0.95NA objective.

### Example 1.16. RNA-Sequencing

Total RNA was extracted from ˜10⁵ cells for each sample (7 different donor-derived CAR⁺ T cell populations, unsorted and migrated separately) using commercially available RNA isolation kit (Macherey-Nagel). The isolated RNA was treated with DNase and the RNA samples were evaluated for quantity and quality with the Qubit (Thermo Fisher Scientific) and Agilent Bioanalyzer High Sensitivity kit. A total of 5 μg of RNA with an RNA Integrity Number (RIN)>7.0 was used to prepare the cDNA libraries with the commercially available SMARTer Stranded Total RNA Seq Kit (Takara), as per the manufacturer's protocols. The libraries were pooled, and a 76 bp paired-end sequencing was performed on an Illumina NextSeq 500 platform using a high output flow cell. After sequencing, the first fifteen bases were trimmed from the 3′ and 5′ sites in all reads to remove the adapter sequences and eliminate the nonuniform distribution caused either by biased selection of reads or contamination of other sequences. The RNA-seq reads were aligned with the HISAT2 (2.0.5) program from Johns Hopkins University and mapped to the human reference genome (GRCh37/hg19).

Differentially expressed genes (DEGs) were identified using DEseq2 tool that tests for differential gene expression based on a model using the negative binomial distribution. The differentially expressed pathways were then identified by the “GAGE” package using the gene sets downloaded from the “Molecular Signature Database” of the Broad Institute and also by using the Gene Set Enrichment Analysis (GSEA) software⁴³.

### Example 1.16. Compound C Inhibition Assays

T cells were incubated with 10 μM dorsomorphin (Sigma Aldrich) for a period of 6-24 hours. The T cells were subsequently used for either motility assays or functional profiling using TIMING assays. Incubation with compound C did not have an impact on T-cell viability.

### Example 1.17. In Vivo Efficacy of CAR⁺ T Cells

On day 0, 7-week-old NOD.Cg-PrkdcscidIl2rgtmlwjl/SzJ (NSG) mice were injected intravenously (i.v.) via a tail vein with 1.5×10⁴ EGFP⁺ ffLuc⁺ NALM-6 cells. Mice (n=10/group) in the 2 treatment cohorts received via tail vein injection (on day 5) of 10⁷ CAR⁺ T cells. One group of mice (n=10) bearing tumor were not treated with T cells. Anesthetized mice underwent bioluminescent imaging (BLI) in an anterior-posterior position using a Xenogen IVIS 100 series system (Caliper Life Sciences) 10 minutes after subcutaneous injection (at neck and shoulder) of 150 μL (200 μg/mouse) freshly thawed aqueous solution of d-Luciferin potassium salt (Caliper Life Sciences) as previously described¹⁶. Photons emitted from NALM-6 xenografts were serially quantified using the Living Image 2.50.1 (Caliper Life Sciences) program. At day 28, five mice in each group were euthanized to evaluate the presence of T cells and tumor cells.

Bone marrow was flushed from the femurs using 30G×½ inch needles (BD, catalog no. 305106) with 2% FBS in PBS. Spleens were disrupted using a syringe in 2% FBS/PBS and passed through a 40 m nylon cell strainer (BD, catalog no. 352340) to obtain a single-cell suspension. Red blood cells from bone marrow, spleen, and peripheral blood were lysed using ACK lysing buffer (Gibco-Invitrogen, A10492) and remaining cells stained for the presence of tumor (CD19 and EGFP) by flow cytometry. The remaining five mice in each group were used to determine the survival curves. In the suboptimal dose model, the mice were treated exactly as above except that at day 5, 2×10⁶ CAR⁺ T cells were injected intravenously.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A method of modifying immune cells,
wherein the method comprises increasing the AMP-activated protein kinase (AMPK) activity of the immune cells to produce modified immune cells,
wherein the increasing enhances the longevity of the modified immune cells.

2. The method of claim 1, wherein the increasing comprises introducing a gene that encodes a protein into the immune cells, wherein the introducing comprises introducing an expression vector into the immune cells, wherein the expression vector encodes and expresses the protein in the immune cells.

3. (canceled)

4. The method of claim 2, wherein the protein is selected from the group consisting of AMPK, Sirtuins, serine/threonine-protein kinase (STK11), CD36, trehalose transporter, derivatives thereof, fusion proteins thereof, subunits thereof, and combinations thereof.

5. The method of claim 2, wherein the protein is AMPK, wherein the AMPK is selected from the group consisting of the alpha subunit of AMPK, AMPK-α1, AMPK-α2, and combinations thereof.

6. (canceled)

7. The method of claim 5, wherein the AMPK comprises a mutation in the kinase active site that renders the expressed AMPK constitutively active.

8. The method of claim 1, wherein the increasing comprises exposing the immune cells to an activator, wherein the exposing occurs for at least 12 hours, and wherein the activator is selected from the group consisting of an AMPK activator, STK11 activator, Sirtuin activator, CD36 activator, Klotho activator, autophagy activator, doxycycline, and combinations thereof.

9-10. (canceled)

11. The method of claim 8, wherein the activator is an AMPK activator selected from the group consisting of sodium butyrate, trehalose, metformin, phenformin, 5-Aminoimidazole-4-carboxamide ribonucleotide (AICAR), aspirin, A-769662, resveratrol, MT 68-73, PF-06409577, PF-249, 5-(5-hydroxy-isoxazol-3-yl)-furan-2-phosphonic acid, and combinations thereof.

12. The method of claim 1, wherein the increasing renders the AMPK activity constitutively active in the modified immune cells, and wherein the enhanced longevity of the modified immune cells is determined by factors selected from the group consisting of enhanced motility, persistent motility, enhanced cellular polarity, enhanced respiratory capacity, an enhanced number of punctate mitochondria, increased mitochondrial mass, reduced conjugation durations, enhanced spare respiratory capacity (SRC), enhanced capacity for oxidative metabolism, and combinations thereof.

13-14. (canceled)

15. The method of claim 1, wherein the immune cells comprise lymphocytes selected from the group consisting of T-cells, CD8⁺ T-cells, CD4⁺ T-cells, NK-cells, B-cells, and combinations thereof.

16. (canceled)

17. The method of claim 1, wherein the immune cells comprise lymphocytes that have been engineered to express one or more immunoreceptors, wherein the one or more immunoreceptors comprise chimeric antigen receptors (CAR).

18. (canceled)

19. The method of claim 1, further comprising a step of obtaining the immune cells from a subject.

20. The method of claim 1, wherein the increasing occurs in vitro, and wherein the method further comprises a step of expanding the modified immune cells in vitro.

21. (canceled)

22. The method of claim 1, further comprising a step of administering the modified immune cells to a subject, wherein the increasing occurs in vivo.

23. (canceled)

24. The method of claim 22, wherein the increasing further comprises changing the diet of the subject, wherein the changing of the diet of the subject comprises at least one of restricting calories, fasting, or adhering to diet conditions that mimic fasting.

25. The method of claim 1, wherein the modified immune cells are utilized to treat a cancer in a subject, wherein the cancer is selected from the group consisting of breast cancer, prostate cancer, pancreatic cancers, glioblastoma, acute lymphocytic leukemia (ALL), chronic myeloid leukemia (CML), lymphomas, leukemias, and combinations thereof.

26. (canceled)

27. A modified immune cell, wherein the modified immune cell has increased AMP-activated protein kinase (AMPK) activity, and wherein the increased AMPK activity enhances the longevity of the modified immune cells.

28. The modified immune cell of claim 27, wherein the modified immune cell comprises an exogenous or endogenous AMPK that is over-expressed or constitutively active, wherein the AMPK is selected from the group consisting of the alpha subunit of AMPK, AMPK-α1, AMPK-α2, and combinations thereof.

29. (canceled)

30. The modified immune cell of claim 27, wherein the enhanced longevity of the modified immune cells is determined by factors selected from the group consisting of enhanced motility, persistent motility, enhanced cellular polarity, enhanced respiratory capacity, an enhanced number of punctate mitochondria, increased mitochondrial mass, reduced conjugation durations, enhanced spare respiratory capacity (SRC), enhanced capacity for oxidative metabolism, and combinations thereof.

31. (canceled)

32. The modified immune cell of claim 27, wherein the modified immune cell comprises lymphocytes, wherein the lymphocytes are selected from the group consisting of T-cells, CD8⁺ T-cells, CD4⁺ T-cells, NK-cells, B-cells, and combinations thereof.

33. (canceled)

34. The modified immune cell of claim 32, wherein the modified immune cell comprises lymphocytes that have been engineered to express one or more immunoreceptors, wherein the one or more immunoreceptors comprise chimeric antigen receptors (CAR).

35. (canceled)
